(19) United States
(12) Patent Application Publication
(10) Pub. No.: US 2010/0255024 A1
(43) Pub. Date: Oct. 7, 2010

(54) COMPOSITION FOR INHIBITION OF TRANSPLANT REJECTION CONTAINING THE PHELLINUS LINTEUS MYCELLIA EXTRACT AS AN ACTIVE INGREDIENT

(75) Inventors: Man Woo Han, Daejeon (KR); Jae Kuk Yoo, Daejeon (KR); Chang-Uk Hur, Daejeon (KR); Hwan-Chul Kim, Daejeon (KR); Jin Pyo Kim, Daejeon (KR)

Correspondence Address: LUCAS & MERCANTI, LLP, 475 PARK AVENUE SOUTH, 15TH FLOOR, NEW YORK, NY, 10016, US

(73) Assignee: HANKOOK PHARM. CO., INC., Nonsan-si, Chungcheongnam-do (KR)

(21) Appl. No.: 12/740,015

(22) PCT Filed: Dec. 7, 2007
(86) PCT No.: PCT/KR2007/006355

§ 371 Date: Apr. 27, 2010

(51) Int. Cl. A61K 36/07; A61P 37/06; A61P 17/00; A61P 17/02

(52) U.S. Cl. 424/195.15

(57) ABSTRACT

Disclosed is a composition for the inhibition of transplant rejection and the prevention and treatment of skin diseases, comprising a Phellinus sp. mycelial extract as an active ingredient. The Phellinus sp. mycelial extract significantly suppresses the production of antibodies to transplants without side effects, such as weight change. Based on natural material, the composition is non-toxic and harmless to the human body and thus can be used as an immunosuppressant for organ transplantation. Also, it stops oozing from sores and is applicable to the prevention and treatment of skin diseases, including atopy, allergic reactions, decubitus ulcers, and smallpox.

### TECHNICAL FIELD

The present invention relates to a composition for inhibition of transplant rejection, comprising a Phellinus sp. mycelial extract as an active ingredient.

### BACKGROUND ART

Transplant rejection occurs when the immune system of the recipient of a transplant attacks the transplanted organ or tissue. Thus, effective suppression of the immune response is known as a main factor determining the success of transplantation. In this regard, the development of immunosuppressive medications has brought about exceptional advances in the transplantation of organs and tissues and the treatment of autoimmune diseases and has made a great contribution to the study of the in vivo mechanism of immune responses to the transplanted organ or tissue.

As described, immunosuppressive drugs were developed to inhibit or attenuate transplant rejection. An example is cyclosporine A (U.S. Pat. No. 4,117,118) produced from Tolypocladium inflatum, a soil fungus. These immunosuppressive drugs not only help realize clinically successful organ transplantation, but also suggest the therapeutic use thereof in treating autoimmune diseases. Even though they are required to act selectively and specifically for T-cells only, conventional immunosuppressive drugs have an influence on a wide range of cellular functions including general signal pathways, causing side effects on other organs, which are healthy (see. S.-H. Lee et al., Korean J. Immunology, 19:375˜389 (1997)). For instance, cyclosporine A is known to show side effects of chronic liver diseases and hypertension after heart transplantation (see: J. E. F. Reynolds, et al., Martindale The Extra Pharmacopoeia, 31^{st} ed., pp. 557-562, Royal Pharmaceutical Society, London, 1996). Many attempts have been made to develop novel immunosuppressive drugs free from side effects. FK-506 has recently been discovered to be an immunosuppressant, and has been commercialized. However, side effects of this drug have also been found (Clin. Transplantation, 11: 237-242 (1997)).

Mushrooms of Phellinus spp. are white-rot fungi belonging to the Phellinaceae family of the Aphylloporales order in the Basidiomycetes class, and have ligneous fruiting bodies. These mushrooms are very rare perennial fungi that parasitize broadleaf trees, including mulberry trees, wild mulberry trees, black oak trees, chestnut trees, oak trees, aspens, willow trees, etc. In ancient Korean and Chinese herb medicine books, the mushrooms of Phellinus spp. are described as various names with excellent medicinal effects. These mushrooms were known among dealers of herbal medicines as legendary medicines from old times because they were difficult to obtain. In recent times, these mushrooms have been identified as enhancing immunity upon chemotherapy for various cancers on the digestive system, including stomach cancer, esophageal cancer, duodenal cancer, colon cancer, rectal cancer, and liver cancer, after resection. Further, they are known to have therapeutic activity on metrorrhagia and leucorrhea, menstrual irregularity, and enterohemorrhage, and activation and detoxication effects on the five viscera and the stomach. However, nowhere has the use of Phellinus spp. as an immunosuppressant been disclosed in the prior art.

Leading to the present invention, intensive and thorough research on an immunosuppressant entailing no side effects, conducted by the present inventors, resulted in the finding that an extract from Phellinus sp. mycelia significantly inhibits the immune response to transplanted organs or tissues.

### DISCLOSURE

### Technical Problem

It is therefore an object of the present invention to provide an immunosuppressive composition, useful in the prevention of transplant rejection, comprising a Phellinus sp. mycelial extract as an active ingredient.

It is another object of the present invention to provide a composition for the prevention and treatment of skin diseases, comprising a Phellinus sp. mycelial extract as an active ingredient.

### Technical Solution

In order to accomplish the objects of the present invention, an immunosuppressive composition comprising a mycelial extract from Phellinus sp. as an active ingredient is provided for the inhibition of transplant injection.

Also, a composition comprising a Phellinus sp. mycelial extract as an active ingredient is provided for the prevention and treatment of skin diseases.

### ADVANTAGEOUS EFFECTS

The Phellinus sp. mycelial extract was found to significantly suppress the production of antibodies to transplants without side effects, such as weight change. Based on a natural material, the composition is non-toxic and harmless to the human body, and thus can be used as an immunosuppressant for organ transplantation. Also, it arrests oozing from sores and is applicable to the prevention and treatment of skin diseases, including atopy, allergic reactions, decubitus ulcers, and smallpox.

### DESCRIPTION OF DRAWINGS

FIG. 1 is a graph showing the levels of antibodies produced against splenocytes transplanted into mice administered with the Phellinus linteus mycelial extract of the present invention or with saline (▪: control, Δ: experimental group).

FIG. 2 is a graph showing changes in the weight of the mice administered with the Phellinus linteus mycelial extract of the present invention and with saline (▪: control, Δ: experimental group).

### BEST MODE

In accordance with an aspect of the present invention, an immunosuppressive composition based on a Phellinus sp. mycelial extract is provided for the inhibition of transplant rejection.

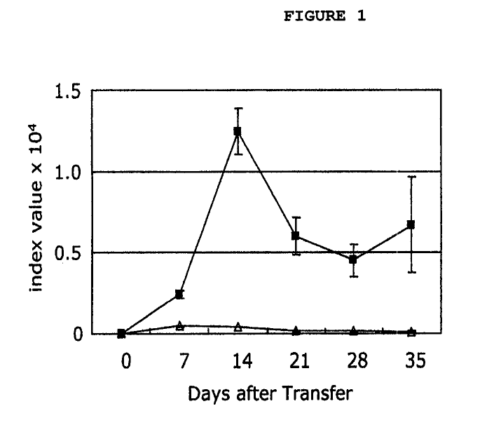

The Phellinus sp. mycelial extract according to the present invention was tested for immunosuppressive effect on smallpox mouse models. After the transplantation of splenocytes thereinto, the animal models were administered with the Phellinus sp. mycelial extract. ELISA analysis on the animal models for the quantification of antibodies to the splenocytes indicated that the Phellinus sp. mycelial extract of the present invention significantly inhibits the production of the antibodies (see FIG. 1). Therefore, the Phellinus sp. mycelial extract according to the present invention can be used as an immunosuppressant for the prevention of transplant rejection.

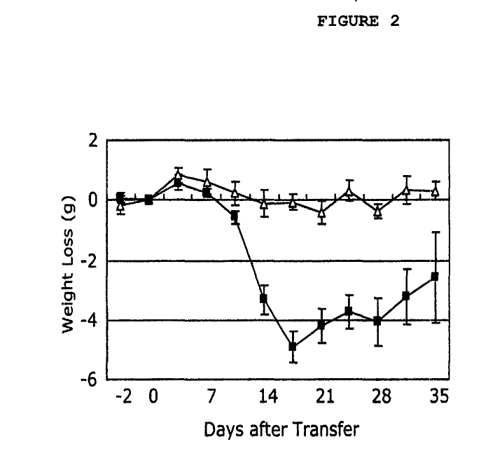

Also, observations were made of whether the Phellinus sp. mycelial extract causes side effects in vivo. Almost no changes were found in the weight of the mice after the transplantation of splenocytes (see FIG. 2). Hence, the Phellinus sp. mycelial extract can be used as an immunosuppressant for the prevention of transplant rejection without the occurrence of side effects, such as changes in weight, upon organ transplantation.

In accordance with another aspect thereof, the present invention provides a composition for the prevention and treatment of skin diseases, comprising a Phellinus sp. mycelial extract as an active ingredient.

The Phellinus sp. mycelial extract according to the present invention was tested for therapeutic activity for skin diseases on smallpox mouse models. After the administration of the Phellinus sp. mycelial extract thereinto, the smallpox mouse models were observed to stop oozing from the smallpox sores. Hence, the Phellinus sp. mycelial extract according to the present invention can be used for the treatment of skin diseases, such as atopy, allergic reactions, decubitus ulcers, smallpox, etc.

The preparation of the Phellinus sp. mycelial extract for the prevention of transplant rejection and the prevention and treatment of skin diseases in accordance with the present invention can be achieved by, but is not limited to, the methods disclosed in Korean Patent Nos. 197446, 174433, and 124853.

Examples of the Phellinus sp. useful in the present invention include Phellinus linteus, Phellinus baumii, and Phellinus igniarius.

When used as medications, the composition may further comprise one or more active ingredients having a function similar to that of the Phellinus sp. mycelial extract.

The Phellinus sp. mycelial extract in accordance with the present invention can be administered orally or non-orally, and may be provided in general medicinal forms. For clinical practice, the Phellinus sp. mycelial extract of the present invention may be used in oral or non-oral forms. It is usually formulated in combination with a diluent or excipient, such as a filler, a thickening agent, a binder, a wetting agent, a disintegrant, a surfactant, etc. Solid agents intended for oral administration of the extract of the present invention may be in the form of tablets, pills, powders, granules, capsules, and the like. In these solid agents, the Phellinus sp. mycelial extract of the present invention is formulated in combination with at least one excipient, such as dextrin, starch, calcium carbonate, sucrose, lactose, or gelatine. In addition, a lubricant, such as magnesium stearate, talc, or the like, may also be added. Liquid agents intended for oral administration include suspensions, internal use solutions, emulsion, syrups, and the like. In addition to a simple diluent, such as water or liquid paraffin, various excipients, such as wetting agents, sweetening agents, aromatics, preservatives, and the like, may be contained in the liquid agents for the oral administration of the extract of the present invention. Also, non-oral dosage forms of the extract of the present invention include sterile aqueous solutions, non-aqueous solutions, suspensions and emulsions, freeze-dried agents, and suppositories. Non-aqueous solutions and suspensions made from propylene glycol, polyethylene glycol, vegetable oils, such as olive oil, and injectable esters such as ethyl oleate may be used. The basic materials of suppositories include Witepsol, macrogol, Tween 61, cacao butter, laurin, glycerol, and gelatin.

The effective dosage of the Phellinus sp. mycelial extract in accordance with the present invention depends on various factors, including the patient's weight, age, gender, state of health, diet, the time of administration, route of administration, excretion rate, etc. For oral administration, the Phellinus sp. mycelial extract in accordance with the present invention may be administered at a dose ranging from 550 to 2,200 mg/day.

For application to the treatment of skin diseases, the Phellinus sp. mycelial extract of the present invention may be used alone or in combination with other therapies, including surgery, radiotherapy, hormonal therapy, chemical therapy and/or biological reaction regulators.

### MODE FOR INVENTION

A better understanding of the present invention may be obtained in light of the following examples, which are set forth to illustrate, but are not to be construed to limit the present invention.

### Example

### Immunosuppression Assay

An immunosuppression test was conducted with the Phellinus sp. mycelial extract of the present invention on smallpox mouse models (obtained from Microbiology Immunology Lab of the Medical College in Keio Univ.).

From two days before the transplantation of splenocytes (Dsg3−/−), five smallpox mice were administered orally with a Phellinus sp. mycelial extract powder (Mesima®; Han Kook Sin Yak) at a dose of 10 mg/kg/day. For 35 days (5 weeks) after the splenocyte transplantation, the Phellinus sp. mycelial extract was orally administered at a dose of 10 mg/kg/day. For a control, physiological saline was used instead of the extract.

Blood samples were taken from the mice 7 days (1 week), 14 days (2 weeks), 21 days (3 weeks), 28 days (4 weeks) and 35 days (5 days) after the transplantation, and were analyzed for the level of antibodies to the splenocytes using ELISA. Throughout the experiment, the weights of the mice were measured every day.

The results are depicted in FIGS. 1 and 2.

In FIG. 1, the levels of the antibodies to the splenocytes are plotted against time for the experimental group and the control group. FIG. 2 shows changes in weight for the experimental group and the control group.

As seen in FIG. 1, the level of antibodies to the transplanted splenocytes in the mice administered with the Phellinus sp. mycelial extract of the present invention was almost zero. From this, it is apparent that the Phellinus sp. mycelial extract according to the present invention effectively inhibits the production of antibodies to transplants.

In FIG. 2, it can be observed that the weight of the control administered with physiological saline slightly increased immediately after the transplantation, but sharply decreased from 7 days after the transplantation, as transplant rejection occurred. In contrast, almost no change was found in the weight of the mice administered with the Phellinus sp. mycelial extract of the present invention. Demonstrated to effectively inhibit the transplant rejection and cause no side effects, such as weight gain, the composition comprising the Phellinus sp. mycelial extract according to the present invention can therefore be used as an immunosuppressive medication applicable for organ or tissue transplantation.

Furthermore, while the control suffered from sores due to smallpox, skin disease was suppressed in the mice administered with the Phellinus sp. mycelial extract of the present invention. Hence, the Phellinus sp. mycelial extract according to the present invention can be applied to the treatment of skin diseases including atopy, allergic reactions, decubitus ulcers, smallpox, etc.

The composition of the present invention can be prepared as described below.

### Preparation Example 1

### Preparation of Pharmaceutical Formulations

1-1. Preparation of Powder

| | |
|---|---|
| Phellinus sp. mycelial extract | 1 g |
| Dextrin | 0.1 g |

The above ingredients were mixed and loaded into an airtight sac to produce powder.

1-2. Preparation of Tablet

| | |
|---|---|
| Phellinus sp. mycelial extract | 500 mg |
| Dextrin | 45 mg |
| Mg Stearate | 5 mg |

These ingredients were mixed and prepared into tablets using a typical tabletting method.

1-3. Preparation of Capsule

| | |
|---|---|
| Phellinus sp. mycelial extract | 500 mg |
| Dextrin | 50 mg |

These ingredients were mixed and loaded into gelatin capsules according to a typical method to produce capsules.

1-4. Preparation of Injection

| Phellinus sp. mycelial extract | 50 | mg/ml |
|---|---|---|
| Diluted HCl BP | added to form pH 3.5 | |
| NaCl BP injection | up to | 1 ml |

The Phellinus sp. mycelial extract was dissolved in a suitable volume of an NaCl BP injection, and the solution was adjusted to a pH of 3.5 with diluted HCl BP and to a desired volume with NaCl BP injection, followed by sufficient mixing. The solution was loaded into transparent 5 ml type I ampules, which were hermetically sealed by melting, followed by autoclaving at 120° C. for 15 min to prepare injections.

1. An immunosuppressive composition for inhibition of transplant injection, comprising a mycelial extract from Phellinus sp. as an active ingredient.

2. The immunosuppressive composition according to claim 1, wherein the Phellinus sp. is Phellinus linteus, Phellinus baumii or Phellinus igniarius.

3. The immunosuppressive composition according to claim 1, wherein the composition is in a dosage form suitable for oral, intravenous or abdominal administration.

4. The immunosuppressive composition according to claim 3, wherein the composition is orally administered at a dose from 550 to 2,200 mg/day.

5. The immunosuppressive composition according to claim 1, wherein the composition suppresses production of antibodies after organ or tissue transplantation, said antibodies causing transplant rejection.

6. A composition for prevention and treatment of skin diseases, comprising a Phellinus sp. mycelial extract as an active ingredient.

7. The composition according to claim 6, wherein the skin diseases are atopy, decubitus ulcer, and/or smallpox.
